(19) United States
(12) Patent Application Publication
(10) Pub. No.: US 2025/0261730 A1
(43) Pub. Date: Aug. 21, 2025

(54) Smart Walking Cane

(71) Applicant: Ying-Chih CHIU, Taichung City (TW)

(72) Inventor: Ying-Chih CHIU, Taichung City (TW)

(21) Appl. No.: 19/059,205

(22) Filed: Feb. 20, 2025

(30) Foreign Application Priority Data
Feb. 21, 2024 (TW) 113106178

(51) Int. Cl. A45B 3/08; A45B 3/04; A45B 9/02

(52) U.S. Cl. CPC A45B 3/08; A45B 3/04; A45B 9/02; A45B 2200/05

(57) ABSTRACT

A smart walking cane may include a smart cane, a smart device, and a smart software. The smart cane has a cane body, and a cane handle is formed on the top of the cane body to be held by the user. The cane body comprises a nine-axis inertial sensor, a pressure sensor, and a photoelectric sensor. The nine-axis inertial sensor has a three-axis accelerometer, a three-axis gyroscope, and a three-axis magnetometer, and the three-axis accelerometer is used to detect the gravitational acceleration of the smart cane along the X, Y, and Z axes, allowing for the detection of movement speed, step count, and whether the user's hand has tremors while using the smart cane.

### FIELD OF THE INVENTION

The present invention relates to a walking cane and more particularly to a smart walking cane with intelligent detection system.

### BACKGROUND OF THE INVENTION

With advancements in modern hygiene and medicine, the average human lifespan has significantly increased. As a result, aging has become a common concern. Due to muscle loss and the decline of hematopoietic function, falls among the elderly can be highly dangerous. After a fall, close monitoring is necessary to prevent damage to vital organs and blood clot formation. Moreover, injuries may not be immediately apparent and could worsen weeks or even months later, so that if the user's physical condition can be detected in advance, fall prevention measures can be implemented early. This would significantly reduce the user's prolonged pain and risks, as well as alleviate the psychological burden, caregiving costs, and valuable time of family members. Additionally, healthcare and medical expenses could be greatly reduced.

With growing health awareness, more people are wearing smartwatches to monitor their physical condition in real time and share data with family members. However, smartwatches are not interactive body detection devices, making it difficult to accurately assess the real-time status of elderly users. For the elderly, having a wearable device that can interactively detect their physical condition is crucial. Since a cane is an essential tool for walking assistance, the idea was conceived to develop a smart cane equipped with interactive detection capabilities. This would enable early awareness of the user's condition, allowing for proactive prevention and reducing the risk of injury. Therefore, there remains a need for a new and improved design for a smart walking cane to overcome the problems presented above.

### SUMMARY OF THE INVENTION

The present invention provides a smart walking cane comprising a smart cane, a smart device, and a smart software. The smart cane has a cane body, and a cane handle is formed on the top of the cane body to be held by the user. The cane body comprises a nine-axis inertial sensor, a pressure sensor, and a photoelectric sensor. The nine-axis inertial sensor has a three-axis accelerometer, a three-axis gyroscope, and a three-axis magnetometer, and the three-axis accelerometer is used to detect the gravitational acceleration of the smart cane along the X, Y, and Z axes, allowing for the detection of movement speed, step count, and whether the user's hand has tremors while using the smart cane. The three-axis gyroscope is used to detect the rotational rate of the smart cane along the X, Y, and Z axes and calculate angles, allowing for the detection of whether the movement frequency remains stable while using the smart cane. The three-axis magnetometer is used to perform yaw correction and direction determination on the six-axis data from the three-axis accelerometer and the three-axis gyroscope, allowing for the detection of whether the movement direction remains stable and in the straight line while using the smart cane. The pressure sensor is used to detect the pressure applied by the user's hand, allowing for the measurement of the force exerted while using the smart cane. The photoelectric sensor is used for optical detection of the user's heart rate variations. When the nine-axis inertial sensor, the pressure sensor, and the photoelectric sensor not detect any values, a warning light and a buzzer are adapted to respectively emit light and sound alerts. The smart cane transmits the detected values from the nine-axis inertial sensor, the pressure sensor, and the photoelectric sensor to the smart device via Bluetooth. The smart device is a smart phone adapted to detect the user's movement distance and step count, and the smart device comprises a GPS, a communication member, and a warning member. The smart device is configured to detect the current location through the GPS, and the smart software is installed in the smart device to be displayed. The notifications and data of the smart device are adapted to be sent to at least one contact through the smart device and the smart software, and the smart software is configured to display the detected and compiled data from the nine-axis inertial sensor, the pressure sensor, the photoelectric sensor, and the smart device, and to analyze and summarize the data to establish the user's normal walking and physical condition parameters. The contact is adapted to access and review the data through the smart software. When the user's walking and physical condition data are abnormal, the smart device is adapted to send the user's current location to the contact through the GPS. The smart device is configured to make a phone call to the contact through the communication member, and the smart device is adapted to activate an alarm sound or send a text message to the contact through the warning member.

Comparing with conventional walking cane, the present invention is advantageous because: When walking, the user relies on the assistance of the smart cane to reduce walking difficulties. The three-axis accelerometer is adapted to detect the user's walking speed, step count, and whether the user's hand has tremors. The three-axis gyroscope is adapted to determine whether the user is swinging the smart cane steadily, allowing for the assessment of movement frequency stability. The three-axis magnetometer is used to perform yaw correction and direction determination on the six-axis data from the three-axis accelerometer and the three-axis gyroscope, so as to detect whether the user's movement direction remains stable and in a straight line, helping to assess the health of the user's legs and the normality of user's sense of balance. The pressure sensor is configured to detect the pressure exerted by the user to determine whether the user's leg strength has decreased, requiring more hand support from the smart cane to assist with walking. The photoelectric sensor is adapted to monitor heart rate to directly assess the user's physical condition. The GPS is configured to record walking locations and to practice tracking to prevent the user from getting lost. The nine-axis inertial sensor utilizes the nine-axis data from the three-axis accelerometer, the three-axis gyroscope, and the three-axis magnetometer, combined with the data measured by the pressure sensor, the photoelectric sensor, and the smart device, to assess the user's walking and physical condition, and the information is displayed through the smart software for the user or the contact to track and provide care. The smart software is configured to compile long-term data and, when abnormalities in the user's walking or physical condition are detected while using the smart cane, the contact is notified through the smart device and smart software to achieve timely alerts and ensure the user's health and safety.

### BRIEF DESCRIPTION OF THE DRAWINGS

FIG. 1 is a partially enlarged perspective view of a smart walking cane of the present invention.

FIG. 2 is a block diagram of the internal structure of the smart walking cane of the present invention.

FIG. 3 is a block diagram of a smart device and smart software of the smart walking cane of the present invention.

FIG. 4 is a schematic diagram showing the connection between the smart device, the smart software and at least a contact of the smart walking cane of the present invention.

FIG. 5 is a first schematic diagram displaying the usage status of the smart software of the present invention, showing the detection data of a nine-axis inertial sensor.

FIG. 6 is a second schematic diagram displaying the usage status of the smart software of the present invention, showing the detection data of a pressure sensor.

FIG. 7 is a third schematic diagram displaying the usage status of the smart software of the present invention, showing the detection and statistical data integration of the smart device and the smart cane.

FIG. 8 is a fourth schematic diagram displaying the usage status of the smart software of the present invention, showing the detection data of a GPS.

### DETAILED DESCRIPTION OF THE INVENTION

The detailed description set forth below is intended as a description of the presently exemplary device provided in accordance with aspects of the present invention and is not intended to represent the only forms in which the present invention may be prepared or utilized. It is to be understood, rather, that the same or equivalent functions and components may be accomplished by different embodiments that are also intended to be encompassed within the spirit and scope of the invention.

Unless defined otherwise, all technical and scientific terms used herein have the same meaning as commonly understood to one of ordinary skill in the art to which this invention belongs. Although any methods, devices and materials similar or equivalent to those described can be used in the practice or testing of the invention, the exemplary methods, devices and materials are now described.

All publications mentioned are incorporated by reference for the purpose of describing and disclosing, for example, the designs and methodologies that are described in the publications that might be used in connection with the presently described invention. The publications listed or discussed above, below and throughout the text are provided solely for their disclosure prior to the filing date of the present application. Nothing herein is to be construed as an admission that the inventors are not entitled to antedate such disclosure by virtue of prior invention.

In order to further understand the goal, characteristics and effect of the present invention, a number of embodiments along with the drawings are illustrated as following:

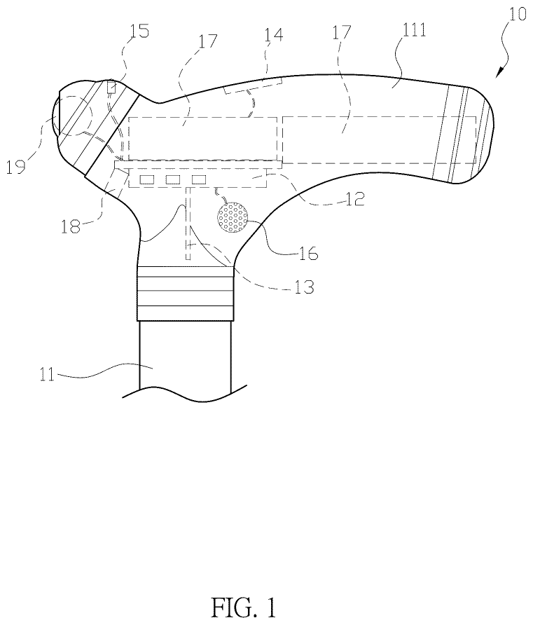

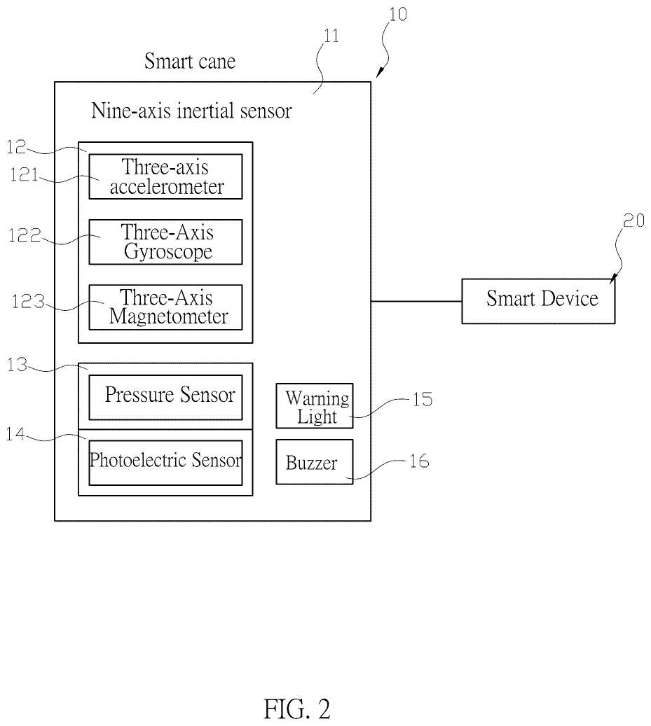

Referring to FIGS. 1 to 3, the present invention provides a smart walking cane comprising a smart cane (10), a smart device (20), and a smart software (30). The smart cane (10) has a cane body (11), and a cane handle (111) is formed on the top of the cane body (11) to be held by the user. Also, the cane body (11) comprises a nine-axis inertial sensor (12), a pressure sensor (13), and a photoelectric sensor (14). The nine-axis inertial sensor (12) has a three-axis accelerometer (121), a three-axis gyroscope (122), and a three-axis magnetometer (123), and the three-axis accelerometer (121) is used to detect the gravitational acceleration of the smart cane (10) along the X, Y, and Z axes, allowing for the detection of movement speed, step count, and whether the user's hand has tremors while using the smart cane (10). The three-axis gyroscope (122) is used to detect the rotational rate of the smart cane (10) along the X, Y, and Z axes and calculate angles, allowing for the detection of whether the movement frequency remains stable while using the smart cane (10). Furthermore, the three-axis magnetometer (123) is used to perform yaw correction and direction determination on the six-axis data from the three-axis accelerometer (121) and the three-axis gyroscope (122), allowing for the detection of whether the movement direction remains stable and in the straight line while using the smart cane (10). In addition, the pressure sensor (13) is used to detect the pressure applied by the user's hand, allowing for the measurement of the force exerted while using the smart cane (10). The photoelectric sensor (14) is used for optical detection of the user's heart rate variations. When the nine-axis inertial sensor (12), the pressure sensor (13), and the photoelectric sensor (14) not detect any values, a warning light (15) and a buzzer (16) are adapted to respectively emit light and sound alerts. The smart cane (10) transmits the detected values from the nine-axis inertial sensor (12), the pressure sensor (13), and the photoelectric sensor (14) to the smart device (20) via Bluetooth.

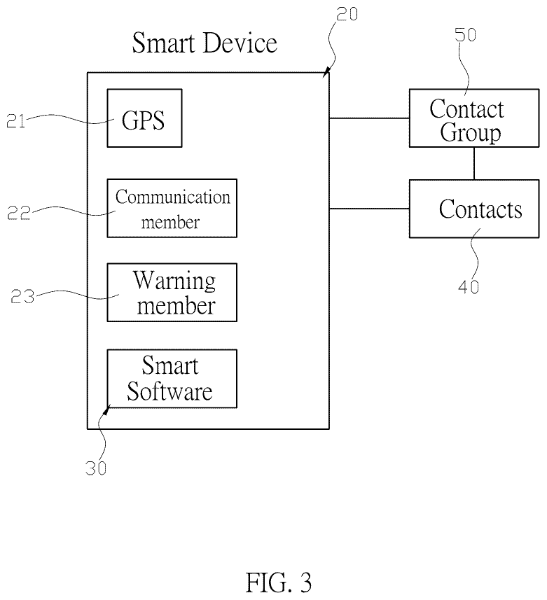

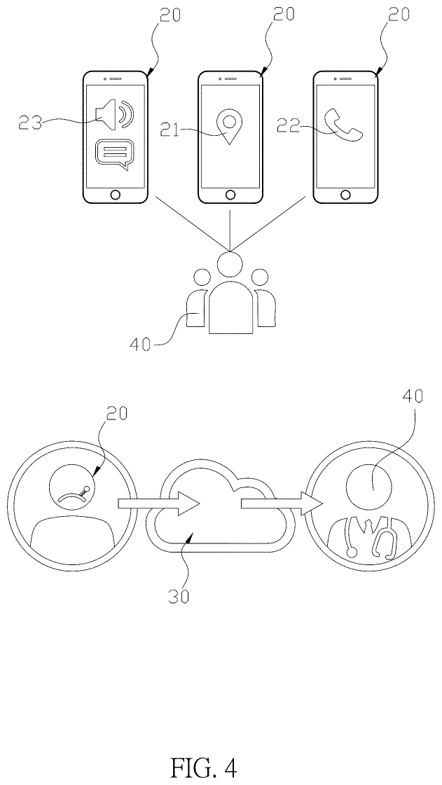

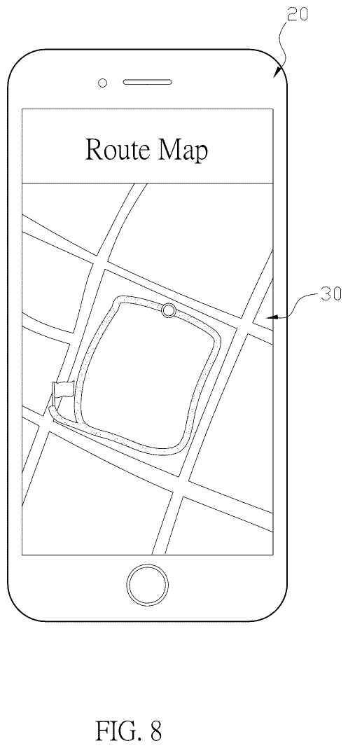

Referring to FIGS. 3 to 8, the smart device (20) is a smart phone adapted to detect the user's movement distance and step count, and the smart device (20) comprises a GPS (21), a communication member (22), and a warning member (23). The smart device (20) is configured to detect the current location through the GPS (21), and the smart software (30) is installed in the smart device (20) to be displayed. The notifications and data of the smart device (20) are adapted to be sent to at least one contact (40) through the smart device (20) and the smart software (30), and the smart software (30) is configured to display the detected and compiled data from the nine-axis inertial sensor (12), the pressure sensor (13), the photoelectric sensor (14), and the smart device (20), and to analyze and summarize the data to establish the user's normal walking and physical condition parameters. Also, the contact (40) is adapted to access and review the data through the smart software (30). When the user's walking and physical condition data are abnormal, the smart device (20) is adapted to send the user's current location to the contact (40) through the GPS (21). Moreover, the smart device (20) is configured to make a phone call to the contact (40) through the communication member (22). Additionally, the smart device (20) is adapted to activate an alarm sound or send a text message to the contact (40) through the warning member (23).

When walking, the user relies on the assistance of the smart cane (10) to reduce walking difficulties. The three-axis accelerometer (121) is adapted to detect the user's walking speed, step count, and whether the user's hand has tremors. The three-axis gyroscope (122) is adapted to determine whether the user is swinging the smart cane (10) steadily, allowing for the assessment of movement frequency stability. The three-axis magnetometer (123) is used to perform yaw correction and direction determination on the six-axis data from the three-axis accelerometer (121) and the three-axis gyroscope (122), so as to detect whether the user's movement direction remains stable and in a straight line, helping to assess the health of the user's legs and the normality of user's sense of balance. The pressure sensor (13) is configured to detect the pressure exerted by the user to determine whether the user's leg strength has decreased, requiring more hand support from the smart cane (10) to assist with walking. The photoelectric sensor (14) is adapted to monitor heart rate to directly assess the user's physical condition. The GPS (21) is configured to record walking locations and to practice tracking to prevent the user from getting lost. The nine-axis inertial sensor (12) utilizes the nine-axis data from the three-axis accelerometer (121), the three-axis gyroscope (122), and the three-axis magnetometer (123), combined with the data measured by the pressure sensor (13), the photoelectric sensor (14), and the smart device (20), to assess the user's walking and physical condition, and the information is displayed through the smart software (30) for the user or the contact (40) to track and provide care. In addition, the smart software (30) is configured to compile long-term data and, when abnormalities in the user's walking or physical condition are detected while using the smart cane (10), the contact (40) is notified through the smart device (20) and smart software (30) to achieve timely alerts and ensure the user's health and safety.

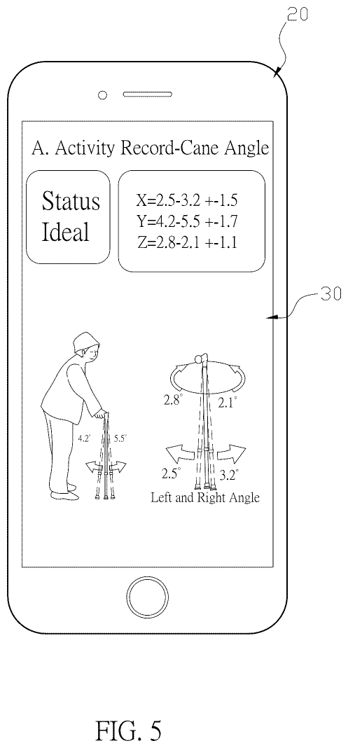

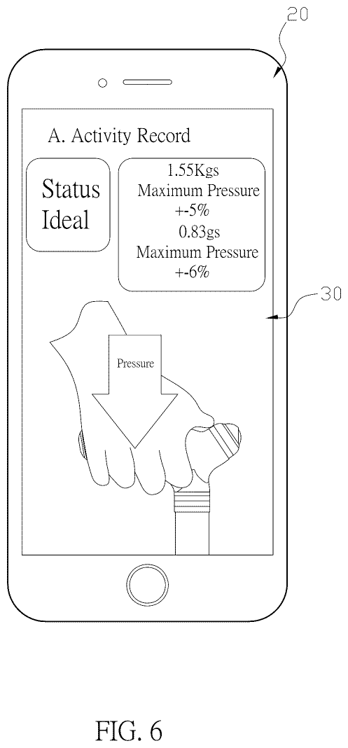

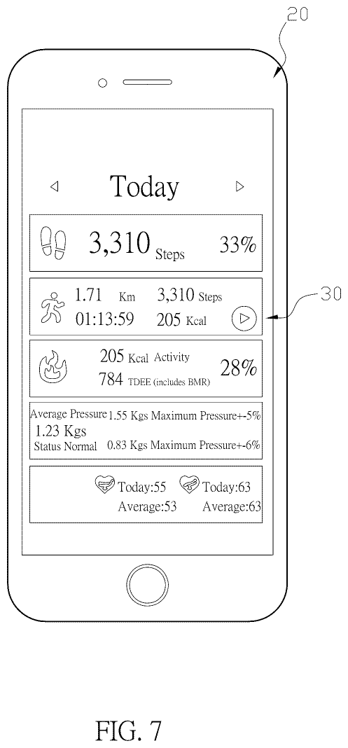

In actual application, referring to FIG. 5, the smart software (30) is adapted to display the complied data detected by the nine-axis inertial sensor (12). Referring to FIG. 6, the smart software (30) is configured to display the complied data detected by the pressure sensor (13). Referring to FIG. 7, the smart software (30) is adapted to display the complied data detected by the pressure sensor (13), the photoelectric sensor (14), and the smart device (20). The smart software (30) is configured to display the compiled data detected by the GPS (21). Based on the design adjustment of the smart software (30), the user and contact (40) are adapted to more conveniently access the integrated detection data through the smart software (30) to track the user's walking and physical condition.

In one embodiment, referring to FIG. 1, the cane body (11) is equipped with at least one built-in battery (17), and the battery is replaceable or recharged by using a charging port (18).

In another embodiment, referring to FIG. 1, the battery (17) supplies power to illumination light (19), so that the smart cane (10) is adapted to provide lighting function, so as to enhance safety in low-light environment.

In still another embodiment, referring to FIG. 1, the pressure sensor (13), and the photoelectric sensor (14) are installed in the cane handle (111), and the pressure sensor (13) is used to detect the force applied by the user when pressing down on the cane handle (111) while the photoelectric sensor (14) uses optical sensing to detect the user's blood vessels in the hand, so as to determine heat rate variations.

In a further embodiment, when the three-axis accelerometer (121) detects a movement speed exceeding the normal speed by 50%, the three-axis gyroscope (122) detects that the smart cane (10) tilts beyond ±45 degrees relative to the ground, the three-axis magnetometer (123) detects no directional movement, the pressure sensor (13) continuously fails to detect pressure, the photoelectric sensor (14) continuously fails to detect a heart rate, and when the nine-axis inertial sensor (12), the pressure sensor (13), and the photoelectric sensor (14) not detect any values for more than 30 seconds, the system is configured to determine that the user has fallen and is at risk. The alert process is adapted to proceed as follows; after 15 seconds, the warning light (15) is adapted to activate and emit a light alarm; after 30 seconds, the buzzer (16) is configured to sound an audible alarm; after 60 seconds, the smart device (20) is adapted to use the warning member (23) to send three consecutive text messages to the contact (40); after 120 seconds, the smart device (20) is configured to use the communication member (22) to make a phone call to the contact (40); and after 180 seconds, the smart device (20) is adapted to use the communication member (22) to call emergency service 911 for assistance. The user is configured to press a stop button (not shown in FIGs.) to confirm that the smart cane (10) is not in use and halt the alarm sequence.

In still a further embodiment, the three-axis accelerometer (121) detects the user's movement speed, while the smart device (20) detects the step count and the GPS (21) tracks the movement distance, and the three sets of data are compiled and analyzed through the smart software (30). During the first three days of using the smart cane (10), the smart software (30) calculates the average time taken and the number of steps per 100 meters, and when the data detected by the three-axis accelerometer (121), the smart device (20), and the GPS (21) remain within a 10% variation range, the smart software (30) is adapted to determine the user under a reasonable fluctuation within normal activity levels. When the variation exceeds the normal range, and the smart software (30) is adapted to determine the user under an abnormal state. In case that the user's average time per 100 meters is 90 seconds and the average step count is 250 steps, so that a range of 81-99 seconds and 225-275 steps is considered a reasonable fluctuation within normal activity levels. On the contrary, when the values exceed the 10% variation range, in case that the time increases to 99-108 seconds or the step count rises to 275-300 steps, indicating the user's movement has slowed down, and the user and the contact (40) are alerted, prompting the user and the contact (40) to arrange a medical check-up as soon as possible to assess the user's health condition.

In a preferred embodiment, the three-axis accelerometer (121) detects the frequency of the user's shaking of the smart cane (10) to assess the user's hand tremor condition, and the three-axis accelerometer (121) is adapted to measure acceleration values along three axes and processes the data through the smart software (30) for analysis. When the data detected by the three-axis accelerometer (121) remains within a 10% variation range, the smart software (30) is adapted to determine the user under a reasonable fluctuation within normal activity levels. Conversely, when the variation exceeds the normal range, and the smart software (30) is adapted to determine the user under an abnormal state. In case that the user's initial average values are X-axis: 10 Km/h, Y-axis: 5 Km/h, Z-axis: 15 Km/h, and later, the values decrease beyond the 10% variation range, such as X-axis: 8 Km/h, Y-axis: 4 Km/h, Z-axis: 12 Km/h, that suggests a reduction in hand tremors and indicates effective rehabilitation and medical treatment leading to improved motor control. On the contrary, when the variation exceeds 10% and the average values of all three axes increases, such as original values: X-axis: 10 Km/h, Y-axis: 5 Km/h, Z-axis: 15 Km/h, and increased values: 12 Km/h, Y-axis: 6 Km/h, Z-axis: 18 Km/h, that suggest a worsening of hand tremors or the new onset of tremors in a previously stable user, so that the user and contact (40) are informed to arrange a medical consultation promptly to assess the underlying hearth condition.

In another preferred embodiment, the three-axis gyroscope (122) is adapted to detect the user's foot movement frequency by measuring the three-axis angular changes of the smart cane (10). The smart software (30) is configured to process the data, and during the first three days of use, the gyroscope data variation within 10% is considered a normal activity range. When the variation exceeds the normal range, and the smart software (30) is adapted to determine the user under an abnormal state. In case that the initial X-axis average ranges from 0 degree to 5 degrees, Y-axis average ranges from +15 degrees to −15 degrees, and Z-axis average ranges from +5 degrees to 0 degree, and a variation within 10% is considered normal, indicating stable foot movement frequency. When the variation exceeds 10%, that suggests the user's foot movement frequency is unstable, and the user and contact (40) are adapted to check the cause of the fluctuating values such as intense exercise or increased activity and to arrange medical consultation for user's physical conditions.

In still another preferred embodiment, the three-axis magnetometer (123) is adapted to detect the movement direction of the smart cane (10), while the GPS (21) provides location data, and the two data sources are compared and analyzed through the smart software (30). In case that the user's movement direction variation stays within 20% that is determined as normal activity. When the variation exceeds 20%, indicating that the user's movement is unstable or disoriented and is determined as abnormal movement. In case that the normal walking direction is north 10 degrees, so that within ±8 degrees to ±12 degrees is in normal ranges, and in case that the subsequent direction deviates between +15 degrees north, that is determined as an abnormal walking pattern. The direction of the smart cane (10) and the movement direction of the GPS (21) show excessive deviation, indicating frequent veering to the left or right instead of moving in a straight line, so that the user and the designated contact (40) is adapted to promptly arrange a medical check-up to assess the user's health condition.

In an advantageous embodiment, the pressure sensor (13) is configured to detect the downward force exerted by the user on the smart cane (10), and the data is analyzed and calculated through the smart software, and when the variation in the user's downward force exceeds 25% for more than 10 minutes, that is considered an abnormal condition.

Referring to FIG. 3, in another advantageous embodiment, multiple contacts (40) are adapted to form at least one contact group (50), and the notifications and data are configured to be sent to contact group (50) through the smart device (20) and the smart software (30).

In still another advantageous embodiment, the three-axis accelerometer (121), commonly known as a G-sensor, is used to measure gravitational acceleration along the X, Y and Z axes (unit: m/s²), and by analyzing the distribution of gravitational acceleration values across the three axes, the inertial forces in each direction are configured to be determined.

In a particular embodiment, the three-axis gyroscope (122), commonly referred to as a Gyro-sensor, measure the rotational rate along X, Y, and XZ axes (unit: rad/s), and in the invention, the three-axis gyroscope (122) is used to detect the swing amplitude and frequency of the smart cane (10), and in case that the swing amplitude is too small or too large, attention is adapted to be given to whether the user's walking frequency and step distance exhibit any abnormalities.

In another particular embodiment, the three-axis magnetometer (123), also known as a geomagnetic sensor or magnetic sensor, is used to measure the strength and direction of magnetic fields and is configured to determine the orientation of a device, which is similar to that of a compass. In actual application, the three-axis magnetometer (123) assists in yaw correction and directional determination by integrating six-axis data from the three-axis accelerometer (121) and the three-axis gyroscope (122). While the three-axis accelerometer (121) provides data to determine the placement and orientation of the smart cane (10), but not measures the speed of flipping or rotation of the smart cane (10) and not detects the instantaneous state of the smart cane (10). Therefore, by incorporating the three-axis gyroscope (122) and performing integral calculation of data from the three-axis accelerometer (121) and the three-axis gyroscope (122), the motion state of the smart cane (10) is obtained. While the integral calculation has minor discrepancies compared to the actual state, and the errors are negligible in the short term but accumulate over, leading to significant deviations. In a six-axis system, after a 360 degrees rotation, the image not returns to its original position, similar to how a person lease their sense of direction. As a result, an accurate reference is needed, and the three-axis magnetometer (123) is introduced to perform corrections and determine the correct direction.

The nine-axis inertial sensor (12) integrates data from the three-axis accelerometer (121), the three-axis gyroscope (122), and the three-axis magnetometer (123) through fusion algorithms to acquire nine-axis data, so as to calculate the corrected orientation of the object. Currently, the nine-axis fusion algorithms include Kalman filter algorithm, particle filter algorithm, and complementary filter algorithm, and for developers, the fusion algorithms essentially take in the nine-axis sensor data along with timestamps and output composite values that are used to comprehensively determine the user's state.

In still another particular embodiment, the pressure sensor (13) is a Load Cell or a weighting sensor, which is a specialized type of force sensor using a combination of a strain gauge and a bridge circuit. When subjected to tensile or compressive forces, the pressure sensor (13) generates a voltage output proportional to the applied force. Load cells come in various types, including compression, tension/compression, dual shear beam, cantilever beam, single-point, and ring load cells. In weighing applications, the Load Cell is calibrated by kilograms (kg). Based on gravitational load, the load cell undergoes elastic deformation, and the strain gauges attached internally convert the deformation proportionally into a voltage signal, which allows for high precision ranging from ±0.01% to ±0.05%, so that the pressure sensor (13) is adapted to accurately record the pressure exerted by the user on the smart cane (10).

During a heartbeat, the heart pumps blood into the arteries, increasing blood flow and reducing the amount of reflected light. Between two consecutive heartbeats, blood flow in the wrist weakens, leading to an increase in reflected light. The photoelectric sensor (14) is configured to measure the time interval between high and low light intensity to determine the heartbeat interval and calculate the heart rate based on the data. Optical heart rate measurement utilizes photoplethysmography (PPG) technology to detect changes in subcutaneous blood volume. Theoretically, PPG signals are adapted to be measured from any part of the body, such as the fingers, earlobes, temples or wrists. While the method is less precise compared to traditional heart rate measurement techniques that analyze the heart's electrical activity, it is more convenient and faster, making it suitable for real-time health monitoring.

In yet another particular embodiment, the smart device (20) is a smartphone, and modern smartphones are equipped with accelerometers and other related sensors on the circuit boards thereof, enabling the smart device (20) to measure acceleration. As a result, when the phone moves on any surface or through space, the movement direction and speed thereof are measured to estimate travel distance, step count, and other relevant data. The user's basic movement data is accurately calculated when the phone is placed in a pocket or secured to the arm during exercise, which allows the smart device (20) to detect the user's travel distance and step count effectively.

In yet a further embodiment, the smart software (30) is an app installed on the smart device (20). When the smart cane (10) is in use, data collected by the nine-axis inertial sensor (12), the pressure sensor (13), and the photoelectric sensor (14), along with the movement distance and step count detected by the smart device (20 and the location data from the GPS (21), are adapted to be displayed on the smart software (30). The walking and health condition thresholds vary based on user parameters such as age, so that the smart software (30) is configured to adjust the standards accordingly. Additionally, by continuously aggregating and analyzing long-term data, the smart software (30) is adapted to assist in detecting abnormal conditions.

Having described the invention by the description and illustrations above, it should be understood that these are exemplary of the invention and are not to be considered as limiting. Accordingly, the invention is not to be considered as limited by the foregoing description, but includes any equivalents.

What is claimed is:

1. A smart walking cane comprising a smart cane, a smart device, and a smart software;
wherein smart cane has a cane body, and a cane handle is formed on the top of the cane body to be held by the user; the cane body comprises a nine-axis inertial sensor, a pressure sensor, and a photoelectric sensor; the nine-axis inertial sensor has a three-axis accelerometer, a three-axis gyroscope, and a three-axis magnetometer, and the three-axis accelerometer is used to detect the gravitational acceleration of the smart cane along the X, Y, and Z axes, allowing for the detection of movement speed, step count, and whether the user's hand has tremors while using the smart cane; the three-axis gyroscope is used to detect the rotational rate of the smart cane along the X, Y, and Z axes and calculate angles, allowing for the detection of whether the movement frequency remains stable while using the smart cane; the three-axis magnetometer is used to perform yaw correction and direction determination on the six-axis data from the three-axis accelerometer and the three-axis gyroscope, allowing for the detection of whether the movement direction remains stable and in the straight line while using the smart cane; the pressure sensor is used to detect the pressure applied by the user's hand, allowing for the measurement of the force exerted while using the smart cane; the photoelectric sensor is used for optical detection of the user's heart rate variations; when the nine-axis inertial sensor, the pressure sensor, and the photoelectric sensor not detect any values, a warning light and a buzzer are adapted to respectively emit light and sound alerts; the smart cane transmits the detected values from the nine-axis inertial sensor, the pressure sensor, and the photoelectric sensor to the smart device via Bluetooth;
wherein the smart device is a smart phone adapted to detect the user's movement distance and step count, and the smart device comprises a GPS, a communication member, and a warning member; the smart device is configured to detect the current location through the GPS, and the smart software is installed in the smart device to be displayed; the notifications and data of the smart device are adapted to be sent to at least one contact through the smart device and the smart software, and the smart software is configured to display the detected and compiled data from the nine-axis inertial sensor, the pressure sensor, the photoelectric sensor, and the smart device, and to analyze and summarize the data to establish the user's normal walking and physical condition parameters; the contact is adapted to access and review the data through the smart software; when the user's walking and physical condition data are abnormal, the smart device is adapted to send the user's current location to the contact through the GPS; the smart device is configured to make a phone call to the contact through the communication member, and the smart device is adapted to activate an alarm sound or send a text message to the contact through the warning member;
wherein the pressure sensor, and the photoelectric sensor are installed in the cane handle, and the pressure sensor is used to detect the force applied by the user when pressing down on the cane handle while the photoelectric sensor uses optical sensing to detect the user's blood vessels in the hand, so as to determine heat rate variations;
wherein the three-axis accelerometer detects the user's movement speed, while the smart device detects the step count and the GPS tracks the movement distance, and the three sets of data are compiled and analyzed through the smart software; during the first three days of using the smart cane, the smart software calculates the average time taken and the number of steps per 100 meters, and when the data detected by the three-axis accelerometer, the smart device, and the GPS remain within a 10% variation range, the smart software is adapted to determine the user under a reasonable fluctuation within normal activity range; when the variation exceeds the normal range, and the smart software is adapted to determine the user under an abnormal state.

2. The smart walking cane of claim 1, wherein when the three-axis accelerometer detects a movement speed exceeding the normal speed by 50%, the three-axis gyroscope detects that the smart cane tilts beyond ±45 degrees relative to the ground, the three-axis magnetometer detects no directional movement, the pressure sensor continuously fails to detect pressure, the photoelectric sensor continuously fails to detect a heart rate, and when the nine-axis inertial sensor, the pressure sensor, and the photoelectric sensor not detect any values for more than 30 seconds, the system is configured to determine that the user has fallen and is at risk; and the alert process is adapted to proceed as follows; after 15 seconds, the warning light is adapted to activate and emit a light alarm; after 30 seconds, the buzzer is configured to sound an audible alarm; after 60 seconds, the smart device is adapted to use the warning member to send three consecutive text messages to the contact; after 120 seconds, the smart device is configured to use the communication member to make a phone call to the contact; and after 180 seconds, the smart device is adapted to use the communication member to call emergency service 911 for assistance; the user is configured to press a stop button to confirm that the smart cane is not in use and halt the alarm sequence.

3. The smart walking cane of claim 1, wherein the three-axis accelerometer detects the frequency of the user's shaking of the smart cane to assess the user's hand tremor condition, and the three-axis accelerometer is adapted to measure acceleration values along three axes and processes the data through the smart software for analysis; when the data detected by the three-axis accelerometer remains within a 10% variation range, the smart software is adapted to determine the user under a reasonable fluctuation within normal activity levels; when the variation exceeds the normal range, and the smart software is adapted to determine the user under an abnormal state.

4. The smart walking cane of claim 1, wherein the three-axis gyroscope is adapted to detect the user's foot movement frequency by measuring the three-axis angular changes of the smart cane; the smart software is configured to process the data, and during the first three days of use, the gyroscope data variation within 10% is considered a normal activity range; when the variation exceeds the normal range, and the smart software is adapted to determine the user under an abnormal state.

5. The smart walking cane of claim 1, wherein the three-axis magnetometer is adapted to detect the movement direction of the smart cane, while the GPS provides location data, and the two data sources are compared and analyzed through the smart software; the user's movement direction variation stays within 20% that is determined as normal activity; when the variation exceeds the normal range, and the smart software is adapted to determine the user under an abnormal state.

6. The smart walking cane of claim 1, wherein the pressure sensor is configured to detect the downward force exerted by the user on the smart cane, and the data is analyzed and calculated through the smart software, and when the variation in the user's downward force exceeds 25% for more than 10 minutes, that is considered an abnormal condition.
